(19) United States
(12) Plant Patent Application Publication
(10) Pub. No.: US 2011/0055988 P1
(43) Pub. Date: Mar. 3, 2011

(54) Mandarin tree named 'Queen'

(50) ;

(75) Inventor: Emilio de Teresa Cortes, El Puig (ES)

(73) Assignee: Angel Teresa Hermanos S.A., Pilar de la Horadada (ES)

(21) Appl. No.: 12/584,258

(22) Filed: Sep. 1, 2009

(51) Int. Cl. A01H 5/00

(52) U.S. Cl. PLT/202

(57) ABSTRACT

‘Queen’ is a new and distinctive mandarin hybrid cultivar, which distinguishes itself by having mid to late season maturity, a large size, distinguished flat shape, easily peeled, a smooth rind texture, white albedo, red color, rich sweet flavor and pleasant aroma. It further distinguishes itself by being seedless or virtually seedless.

### LATIN NAME OF THE GENUS AND SPECIES

The mandarin cultivar of this invention is botanically identified as Citrus reticulata.

### VARIETY DENOMINATION

The variety denomination is ‘Queen’.

### BACKGROUND OF THE INVENTION

This invention relates to a new and distinctive mandarin hybrid cultivar designated ‘Queen’ which was developed in Spain. It is distinguished from other cultivars by producing fruit that combines mid to late season maturity, large size, distinguished flat shape, easily peeled, smooth rind texture, white albedo, red color, rich sweet flavor and pleasant aroma. It further distinguishes itself by being seedless or virtually seedless.

### BRIEF SUMMARY OF THE INVENTION

Queen mandarin is a natural hybrid of a ‘M. Satsuma’ seed that appeared in Pilar de la Horadada, Alicante, Spain in 1987. It was grafted over Macrophylla, Volkameriana, Carrizo and Cleopatra rootstocks. Fruit production and evaluation began in 1995. Trials with cross pollination have been made with ‘Orogrande’, ‘Oronules’, ‘Ortanique’, ‘Clemenpons,’ ‘Fortune’ and ‘Valencia-Late’. Only the trials with ‘Fortune’ have occasionally produced some seed in the fruit of the ‘Queen’ mandarin. In 2006, budwood was sent to a center in Valencia, Spain, to clean the variety of the tristeza virus. In 2007, four trees certified as tristeza-free were kept in a breeding block of the center for further propagation and four trees were sent to a breeding block in Pilar de la Horadada, Alicante, Spain. Fruit production of those trees commenced in 2009.

### BRIEF DESCRIPTION OF THE DRAWINGS

The figures depict various characteristics of ‘Queen’.

FIG. 1 shows the ‘Queen’ mandarin flower.

FIG. 2 shows the ‘Fortune’ mandarin flower.

FIG. 3a shows the fruit of a three year old ‘Queen’ tree in week three, grafted over a 20 year old Cleopatra rootstock with Valencia Late.

FIG. 3b shows a close-up view of the fruit of ‘Queen’ in week three.

FIG. 4a shows the peeled fruit of ‘Queen’ mandarin with navel.

FIG. 4b shows the fruit of a three year old grafted ‘Queen’ mandarin during week 13.

FIG. 5 shows a view of a three year old grafted ‘Queen’ mandarin tree over a 20 year old rootstock of Cleopatra (Valencia Late)

FIG. 6 shows a four year old grafted ‘Queen’ mandarin tree over a 20 year old rootstock of Cleopatra (Valencia Late)

FIG. 7a shows a cross section of representative mid-season ‘Queen’ fruit.

FIG. 8a shows a top view (stem end) of a typical fruit of ‘Queen’.

FIG. 8b shows a bottom view (blossom end or tip) of a typical fruit of ‘Queen’.

### DETAILED DESCRIPTION OF THE INVENTION

The following is a detailed description of the new Citrus reticulata, ‘Queen’, based upon observation of plants grown in Alicante, Spain. The color designations being according to The R.H.S. Colour Chart published by The Royal Horticultural Society of London, England.

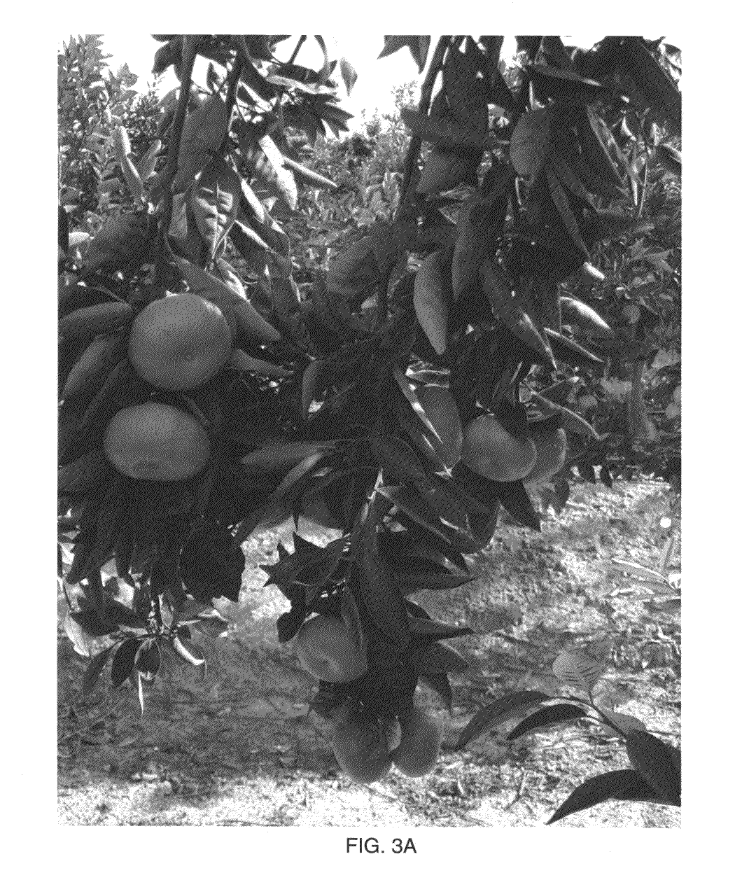

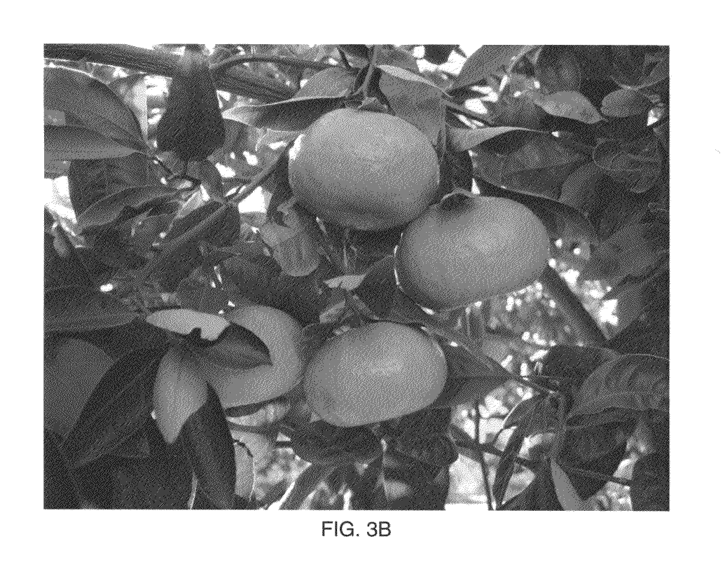

Fruit of ‘Queen’ mandarins are virtually seedless and do not cross-pollinate with other mandarins as it is auto incompatible. ‘Queen’ mandarin is a mid to late season variety, harvested between the beginning of January to the end of April in Pilar de la Horadada, Alicante, Spain. The fruit is very easy to peel, with a flat shape and having a diameter between 58-68 mm. The fruit has around 49% juice content, is very homogeneous, and has a red fruit skin color (see FIG. 3a), with a typical flat shape (see FIG. 3b). The oil glands have a pleasant aroma. At maturity the ‘Queen’ mandarin fruit has around 1.05% of acid concentration and 12° Brix. At the beginning of January, the ‘Queen’ mandarin has around 1.25% of acid concentration and 11.5° Brix over Macrophylla rootstock. By the end of February, the ‘Queen’ mandarin has around 1.15% of acid concentration and 13.5° Brix over Cleopatra rootstock. By mid-April, the ‘Queen’ mandarin has around 1.00% of acid concentration and 15° Brix over Cleopatra rootstock.

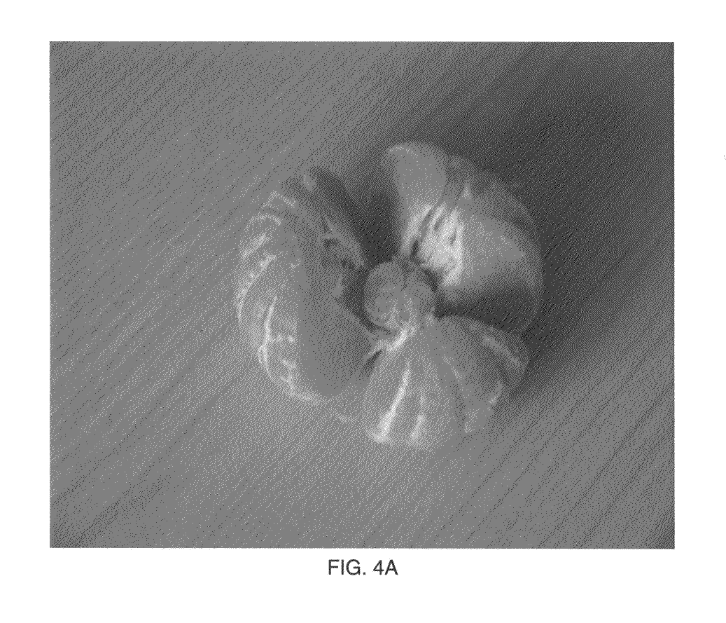

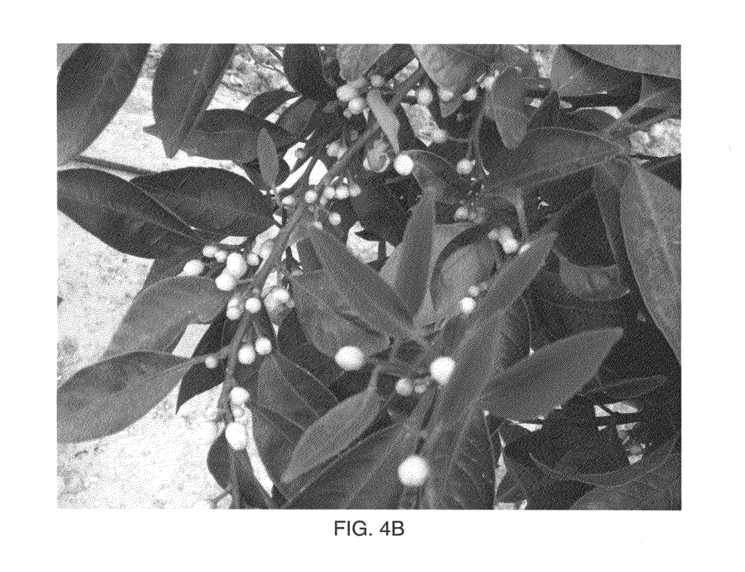

The flavor is sweet and lightly acidic. The pulp has a smooth texture and the segments have an easy-eating texture. Some ‘Queen’ fruit have navels that differ from 1 to 5 mm in diameter and develop inside as a typical navel orange does (See FIG. 4a). Usually, between 14 and 16 segments are found in a ‘Queen’ mandarin fruit. Growing conditions are easy to manage as they do not need pruning and treatments to produce a good yield.
Comparison with existing mandarins:

The ‘Queen’ mandarin variety differs from its parents in harvesting period. ‘Satsuma’ is harvested in October through December and ‘Queen’ is harvested in January through April, in Pilar de la Horadada, Alicante, Spain. ‘Satsuma’ has a puffy skin, while ‘Queen’ has a skin that is easy to peel, but not puffy. ‘Satsuma’ has an orange color, while ‘Queen’ has a red color. ‘Satsuma’ has low level acidity during harvesting period, between 0.90 and 0.60, while ‘Queen’ has an acid level of 1.25 and 1.00 during harvesting.

‘Queen’ mandarin differs from main mid to late season cultivars because trees of ‘Queen’ do not show alternate bearing and do not need to be pruned. ‘Queen’ is virtually seedless, sweeter and more acidic than other varieties. ‘Queen’ mandarin's resistance to heat and cold is similar to ‘Clementine’.
Trees, foliage and flowers:

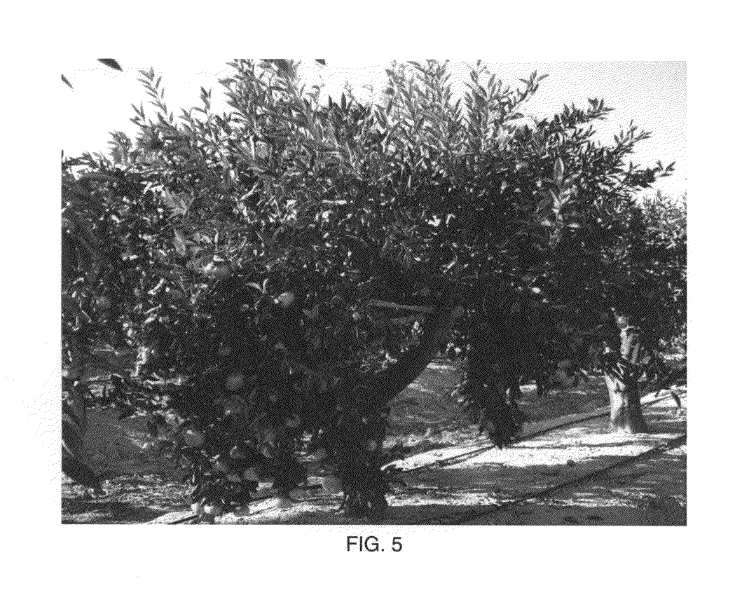

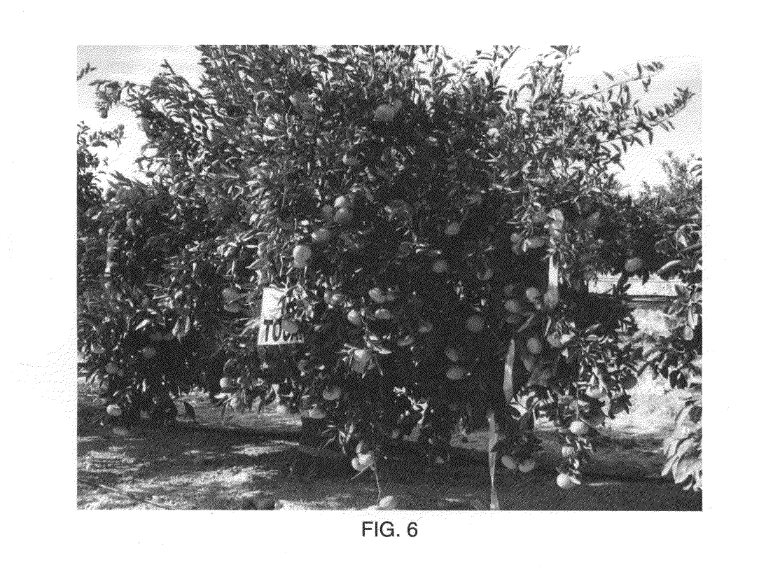

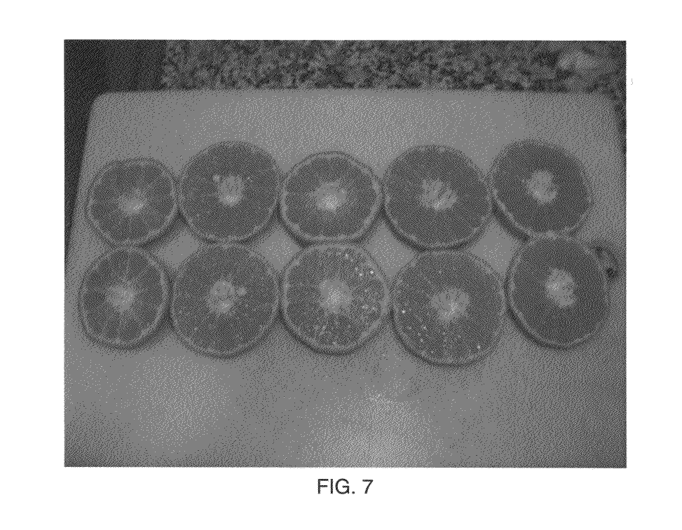

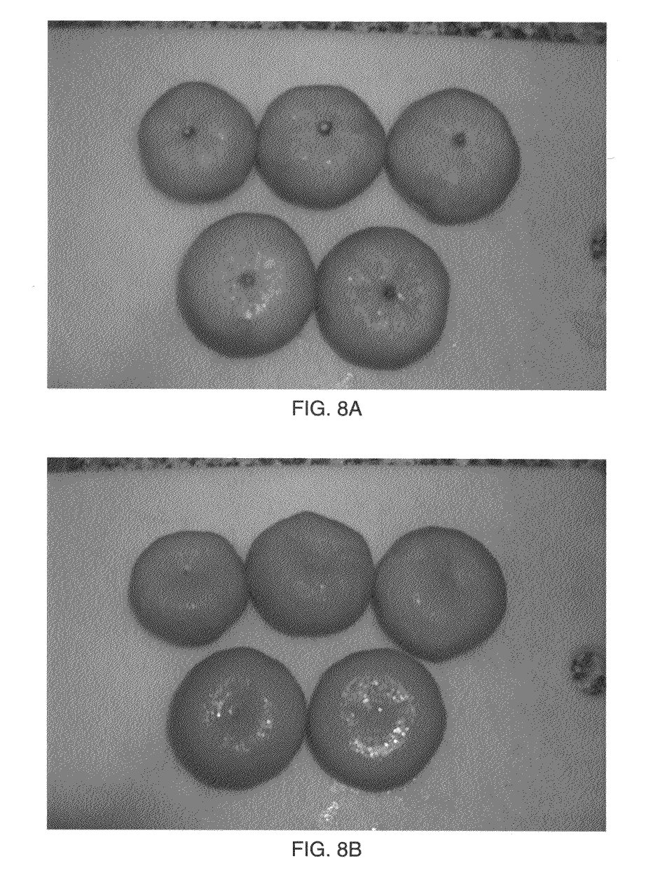

The ‘Queen’ mandarin tree has a drooping growth habit, obloid shape and is without spines. The bark of the trunk has a green and brown color and the branches tend to curve down. The tree is vigorous with dark green leaves (See FIGS. 5-6). A typical five-year old ‘Queen’ mandarin tree has an average height of 2.60 m and an average width of 2.20 m. A typical truck diameter is 7 cm at 30 cm about the ground.

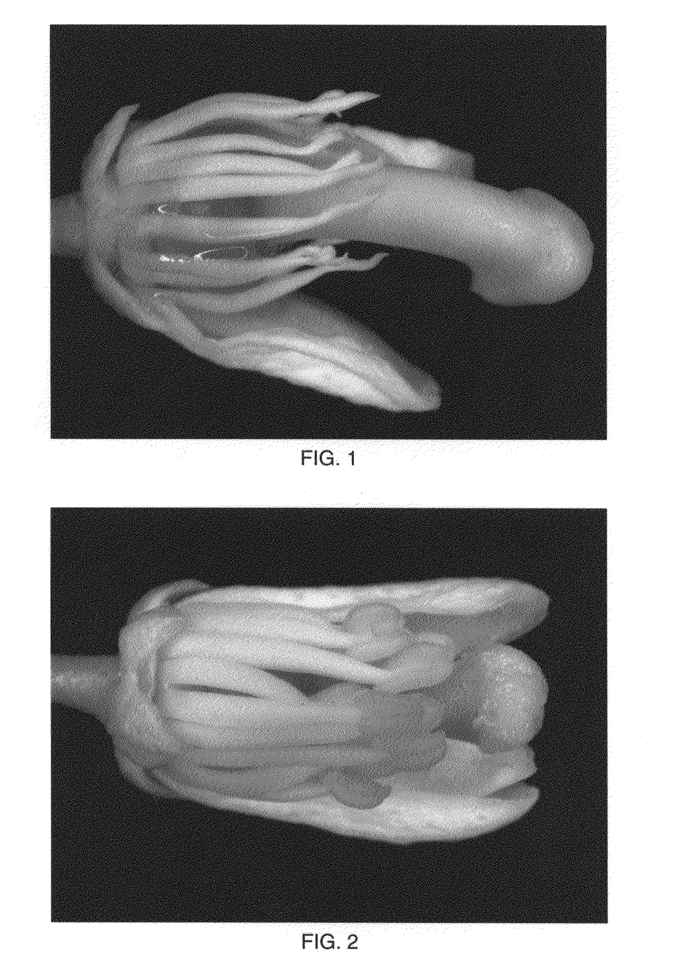

Flowering occurs at the end of March to April in. Pilar de la Horadada, Alicante, Spain (See, FIG. 4b) The flowers have no pollen and are hermaphrodite. Open flowers are white (RHS color: 155 C) and have five petals. The flowers do not have anthers and cannot produce pollen (See, FIGS. 1, 2). The ratio of petal length and width, and stamen length, is medium.

Leaf length on average is 10.2 cm, with a medium leaf width of 4.0 cm on average, and a large leaf ratio (2.55). There is no leaf blade or spine. The leaf has a straight shape in the cross section and an acute shape of apex. The length of the petiole is medium and wings are absent. Leaf color is typically dark green, RHS 139 A.
Fruiting, fruit and production characteristics:

‘Queen’ mandarin fruit is typically harvested middle of January to end of April in Pilar de la Horadada, Alicante, Spain. Fruit production is similar to its parent. Fruit production of a five year old ‘Queen’ mandarin tree grafted in a plantation over Cleopatra rootstock is on average 75 kg per tree.

The ‘Queen’ mandarin fruit has a short height, on average 45.2 mm, and a medium to large diameter, on average 65.9 mm. The color of the fruit surface is typically orange-red, RHS 30A. It has a flattened general shape, absence of neck and presence of depression at stalk end. There are an intermediate number of radial grooves at the stock end, absent presence of collar, presence of depression at distal end, and complete grooved presence of the areola. Occasionally there is the presence of a navel. The fruit rind has medium thickness, medium strength and medium oiliness, with a weak adherence to flesh, white color and loose density of albedo.

The fruit flesh has a medium orange color, large core diameter, and an average of 16 well developed segments: Specifically, the typical color of the mature internal fruit flesh is RHS 30D. The segment walls have a strong strength and there is an intermediate presence of a navel internally. The fruit, while virtually seedless, will occasionally have some seeds and the variety does not require pollination. The fruit flavor is sweet and lightly acidic, having a mandarin aroma, with soft pulp texture and thicker segment texture than a ‘Clementine’.

Results of storage trials have indicated that the first significant signs of fruit deterioration has been found at day 37, with no post harvesting treatments (as imazalil) at +5 Celsius. Fruit of the trial had a Brix of 13 and acid of 1.05%.

No susceptibilities to plant or fruit diseases or to pests beyond those normally associated with citrus species have been observed. Specifically, ‘Queen’ mandarin is tolerant to Citrus Tristeza Virus (CTV) and Alternaria species. Further, Aphyds, mites and scales affect this variety similar to ‘Clementines’. Like all varieties, only summer treatments for pests are needed.

What is claimed is:

1. A new and distinct cultivar of mandarin tree having the characteristics substantially as described and illustrated herein.
